(19) United States
(12) Patent Application Publication
(10) Pub. No.: US 2003/0105294 A1
(43) Pub. Date: Jun. 5, 2003

(54) ENHANCING THE CIRCULATING HALF LIFE OF ANTIBODY-BASED FUSION PROTEINS

(76) Inventors: STEPHEN GILLIES, CARLISLE, MA (US); KIN-MING LO, LEXINGTON, MA (US); YAN LAN, BELMONT, MA (US); JOHN WESOLOWSKI, WEYMOUTH, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/256,156

(22) Filed: Feb. 24, 1999

Related U.S. Application Data
(60) Provisional application No. 60/075,887, filed on Feb. 25, 1998.

(51) Int. Cl. C07K 16/46; C07K 14/54

(52) U.S. Cl. 530/351; 530/391.1

(57) ABSTRACT

Disclosed are methods for the genetic construction and expression of antibody-based fusion proteins with enhanced circulating half-lives. The fusion proteins of the present invention lack the ability to bind to immunoglobulin Fc receptors, either as a consequence of the antibody isotype used for fusion protein construction, or through directed mutagenesis of antibody isotypes that normally bind Fc receptors. The fusion proteins of the present invention may also contain a functional domain capable of binding an immunoglobulin protection receptor.

### CROSS REFERENCE TO RELATED APPLICATION

This incorporates by reference, and claims priority to and the benefit of, U.S. Provisional Patent Application Ser. No. 60/075,887 which was filed on Feb. 25, 1998.

### FIELD OF THE INVENTION

The present invention relates generally to fusion proteins. More specifically, the present invention relates to methods of enhancing the circulating half-life of antibody-based fusion proteins.

### BACKGROUND OF THE INVENTION

The use of antibodies for treatment human disease is well established and has become more sophisticated with the introduction of genetic engineering. Several techniques have been developed to improve the utility of antibodies. These include: (1) the generation of monoclonal antibodies by cell fusion to create “hyridomas”, or by molecular cloning of antibody heavy (H) and light (L) chains from antibody-producing cells; (2) the conjugation of other molecules to antibodies to deliver them to preferred sites in vivo, e.g., radioisotopes, toxic drugs, protein toxins, and cytokines; (3) the manipulation of antibody effector functions to enhance or diminish biological activity; (4) the joining of other protein such as toxins and cytokines with antibodies at the genetic level to produce antibody-based fusion proteins; and (5) the joining of one or more sets of antibody combining regions at the genetic level to produce bi-specific antibodies.

When proteins are joined together through either chemical or genetic manipulation, it is often difficult to predict what properties that the end product will retain from the parent molecules. With chemical conjugation, the joining process may occur at different sites on the molecules, and generally results in molecules with varying degrees of modification that can affect the function of one or both proteins. The use of genetic fusions, on the other hand, makes the joining process more consistent, and results in the production of consistent end products that retain the function of both component proteins. See, for example, Gillies et al, PROC. NATL. ACAD. Sci. USA 89: 1428-1432 (1992); and U.S. Pat. No. 5,650,150.

However, the utility of recombinantly-produced antibody-based fusion proteins may be limited by their rapid in vivo clearance from the circulation. Antibody-cytokine fusion proteins, for example, have been shown to have a significantly lower in vivo circulating half-life than the free antibody. When testing a variety of antibody-cytokine fusion proteins, Gillies et al. reported that all of the fusion proteins tested had an α phase (distribution phase) half-life of less than 1.5 hour. Indeed, most of the antibody-based fusion protein were cleared to 10% of the serum concentration of the free antibody by two hours. See, Gillies et al., BIOCONJ. CHEM. 4: 230-235 (1993). Therefore, there is a need in the art for methods of enhancing the in vivo circulating half-life of antibody-based fusion proteins.

### SUMMARY OF THE INVENTION

A novel approach to enhancing the in vivo circulating half-life of antibody-based fusion proteins has now been discovered. Specifically, the present invention provides methods for the production of fusion proteins between an immunoglobulin with a reduced binding affinity for an Fc receptor, and a second non-immunoglobulin protein. Antibody-based fusion proteins with reduced binding affinity for Fc receptors have a significantly longer in vivo circulating half-life than the unlinked second non-immunoglobulin protein.

IgG molecules interact with three classes of Fc receptors (FcR) specific for the IgG class of antibody, namely FcγRI, FcγRII and FcγRIII. In preferred embodiments, the immunoglobulin (Ig) component of the fusion protein has at least a portion of the constant region of an IgG that has a reduced binding affinity for at least one of FcγRI, FcγRII or FcγRIII.

In one aspect of the invention, the binding affinity of fusion proteins for Fc receptors is reduced by using heavy chain isotypes as fusion partners that have reduced binding affinity for Fc receptors on cells. For example, both human IgG1 and IgG3 have been reported to bind to FcRγI with high affinity, while IgG4 binds 10-fold less well, and IgG2 does not bind at all. The important sequences for the binding of IgG to the Fc receptors have been reported to be located in the CH2 domain. Thus, in a preferred embodiment, an antibody-based fusion protein with enhanced in vivo circulating half-life is obtained by linking at least the CH2 domain of IgG2 or IgG4 to a second non-immunoglobulin protein.

In another aspect of the invention, the binding affinity of fusion proteins for Fc receptors is reduced by introducing a genetic modification of one or more amino acid in the constant region of the IgG1 or IgG3 heavy chains that reduces the binding affinity of these isotypes for Fc receptors. Such modifications include alterations of residues necessary for contacting Fc receptors or altering others that affect the contacts between other heavy chain residues and Fc receptors through induced conformational changes. Thus, in a preferred embodiment, an antibody-based fusion protein with enhanced in vivo circulating half-life is obtained by first introducing a mutation, deletion, or insertion in the IgG1 constant region at one or more amino acid selected from Leu₂₃₄, Leu₂₃₅, Gly₂₃₆, Gly₂₃₇, Asn₂₉₇, and Pro₃₃₁, and then linking the resulting immunoglobulin, or portion thereof, to a second non-immunoglobulin protein. In an alternative preferred embodiment, the mutation, deletion, or insertion is introduced in the IgG3 constant region at one or more amino acid selected from Leu₂₈₁, Leu₂₈₂, Gly₂₈₃, Gly₂₈₄, Asn₃₄₄, and Pro₃₇₈, and the resulting immunoglobulin, or portion thereof, is linked to a second non-immunoglobulin protein. The resulting antibody-based fusion proteins have a longer in vivo circulating half-life than the unlinked second non-immunoglobulin protein.

In a preferred embodiment, the second non-immunoglobulin component of the fusion protein is a cytokine. The term “cytokine” is used herein to describe proteins, analogs thereof, and fragments thereof which are produced and excreted by a cell, and which elicit a specific response in a cell which has a receptor for that cytokine. Preferably, cytokines include interleukins such as interleukin-2 (IL-2), hematopoietic factors such as granulocyte-macrophage colony stimulating factor (GM-CSF), tumor necrosis factor (TNF) such as TNFα, and lymphokines such as lymphotoxin. Preferably, the antibody-cytokine fusion protein of the present invention displays cytokine biological activity.

In an alternative preferred embodiment, the second non-immunoglobulin component of the fusion protein is a ligand-binding protein with biological activity. Such ligand-binding proteins may, for example, (1) block receptor-ligand interactions at the cell surface; or (2) neutralize the biological activity of a molecule (e.g., a cytokine) in the fluid phase of the blood, thereby preventing it from reaching its cellular target. Preferably, ligand-binding proteins include CD4, CTLA-4, TNF receptors, or interleukin receptors such as the IL-1 and IL-4 receptors. Preferably, the antibody-receptor fusion protein of the present invention displays the biological activity of the ligand-binding protein.

In yet another alternative preferred embodiment, the second non-immunoglobulin component of the fusion protein is a protein toxin. Preferably, the antibody-toxin fusion protein of the present invention displays the toxicity activity of the protein toxin.

In a preferred embodiment, the antibody-based fusion protein comprises a variable region specific for a target antigen and a constant region linked through a peptide bond to a second non-immunoglobulin protein. The constant region may be the constant region normally associated with the variable region, or a different one, e.g., variable and constant regions from different species. The heavy chain can include a CH1, CH2, and/or CH3 domains. Also embraced within the term “fusion protein” are constructs having a binding domain comprising framework regions and variable regions (i.e., complementarity determining regions) from different species, such as are disclosed by Winter, et al., GB 2,188, 638. Antibody-based fusion proteins comprising a variable region preferably display antigen-binding specificity. In yet another preferred embodiment, the antibody-based fusion protein further comprises a light chain. The invention thus provides fusion proteins in which the antigen-binding specificity and activity of an antibody are combined with the potent biological activity of a second non-immunoglobulin protein, such as a cytokine. A fusion protein of the present invention can be used to deliver selectively the second non-immunoglobulin protein to a target cell in vivo so that the second non-immunoglobulin protein can exert a localized biological effect.

In an alternative preferred embodiment, the antibody-based fusion protein comprises a heavy chain constant region linked through a peptide bond to a second non-immunoglobulin protein, but does not comprise a heavy chain variable region. The invention thus further provides fusion proteins which retain the potent biological activity of a second non-immunoglobulin protein, but which lack the antigen-binding specificity and activity of an antibody.

In preferred embodiments, the antibody-based fusion proteins of the present invention further comprise sequences necessary for binding to Fc protection receptors (FcRp), such as beta-2 microglobulin-containing neonatal intestinal transport receptor (FcRn).

In preferred embodiments, the fusion protein comprises two chimeric chains comprising at least a portion of a heavy chain and a second, non-Ig protein are linked by a disulfide bond.

The invention also features DNA constructs encoding the above-described fusion proteins, and cell lines, e.g., myelomas, transfected with these constructs.

These and other objects, along with advantages and features of the invention disclosed herein, will be made more apparent from the description, drawings, and claims that follow.

### BRIEF DESCRIPTION OF THE DRAWINGS

The foregoing and other objects, features, and advantages of the present invention, as well as the invention itself, may be more fully understood from the following description of preferred embodiments, when read together with the accompanying drawings, in which:

FIG. 1 is a homology alignment of the amino acid sequences of the constant region of Cγ1 and Cγ3, aligned to maximize amino acid identity, and wherein non-conserved amino acids are identified by boxes;

FIG. 2 is a homology alignment of the amino acid sequences of constant region of Cγ1, Cγ2, and Cγ4, aligned to maximize amino acid identity, and wherein non-conserved amino acids are identified by boxes;

FIG. 3 is a diagrammatic representation of a map of the genetic construct encoding an antibody-based fusion protein showing the relevant restriction sites;

FIG. 4 is a bar graph depicting the binding of antibody hu-KS-¼ and antibody-based fusion proteins, hu-KSγ1-IL2 and hu-KSγ4-IL2, to Fc receptors on mouse J774 cells in the presence (solid bars) or absence (stippled bars) of an excess of mouse IgG;

FIG. 5 is a line graph depicting the in vivo plasma concentration of total antibody (free antibody and fusion protein) of hu-KSγ1-IL2 (closed diamond) and hu-KSγ4-IL2 (closed triangle) and of intact fusion protein of hu-KSγ1-IL2 (open diamond) and hu-KSγ4-IL2 (open triangle) as a function of time;

FIG. 6 is a diagrammatic representation of protocol for constructing an antibody-based fusion protein with a mutation that reduces the binding affinity to Fc receptors;

FIG. 7 is a line graph depicting the in vivo plasma concentration of intact fusion protein of hu-KSγ1-IL2 (⋄); mutated hu-KSγ1-IL2 (□) and hu-KSγ4-IL2 (Δ) as a function of time.

### DETAILED DESCRIPTION OF THE INVENTION

It has now been discovered that fusing a second protein, such as a cytokine, to an immunoglobulin may alter the antibody structure, resulting in an increase in binding affinity for one or more of the cell-bound Fc receptors and leading to a rapid clearance of the antibody-based fusion protein from the circulation. The present invention describes antibody-based fusion proteins with enhanced in vivo circulating half-lives and involves producing, through recombinant DNA technology, antibody-based fusion proteins with reduced binding affinity for one or more Fc receptor.

First, an antibody-based fusion protein with an enhanced in vivo circulating half-life can be obtained by constructing a fusion protein with isotypes having reduced binding affinity for a Fc receptor, and avoiding the use of sequences from antibody isotypes that bind to Fc receptors. For example, of the four known IgG isotypes, IgG1 (Cγ1) and IgG3 (Cγ3) are known to bind FcRγI with high affinity, whereas IgG4 (Cγ4) has a 10-fold lower binding affinity, and IgG2 (Cγ2) does not bind to FcRγI. Thus, an antibody-based fusion protein with reduced binding affinity for a Fc receptor could be obtained by constructing a fusion protein with a Cγ2 constant region (Fc region) or a Cγ4 Fc region, and avoiding constructs with a Cγ1 Fc region or a Cγ3 Fc region.

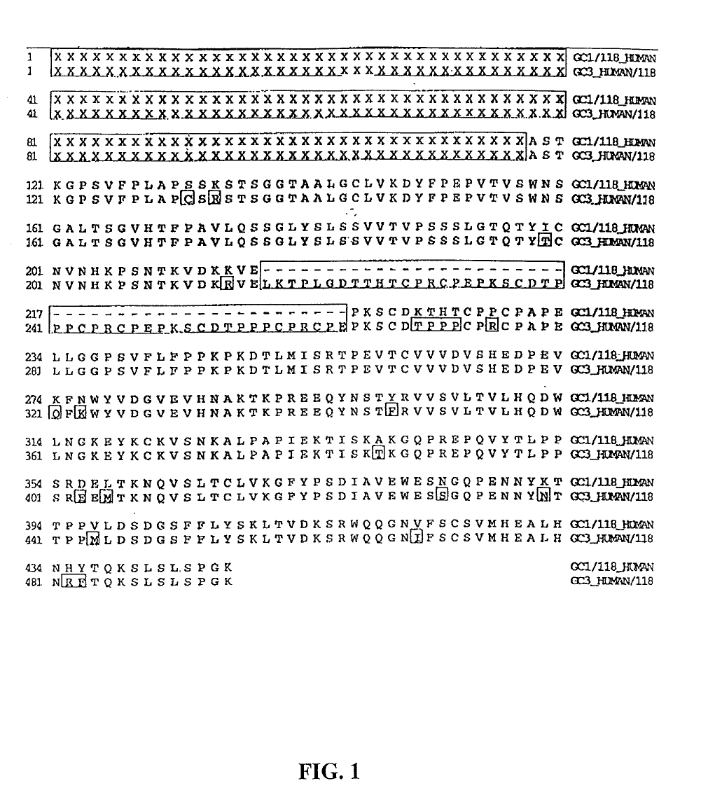

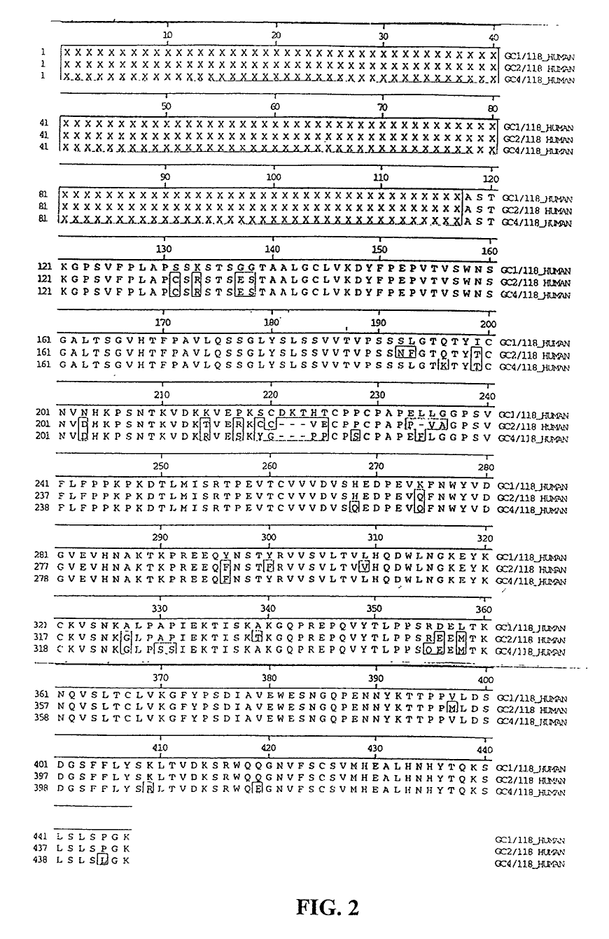

Second, an antibody-based fusion protein with an enhanced in vivo circulating half-life can be obtained by modifying sequences necessary for binding to Fc receptors in isotypes that have binding affinity for an Fc receptor, in order to reduce or eliminate binding. As mentioned above, IgG molecules interact with three classes of Fc receptors (FcR), namely FcγRI, FcγRII, and FcγRIII. Cγ1 and Cγ3 bind FcRγI with high affinity, whereas Cγ4 and Cγ2 have reduced or no binding affinity for FcRγI. A comparison of the Cγ1 and Cγ3 indicates that, with the exception of an extended hinge segment in Cγ3, the amino acid sequence homology between these two isotypes is very high. This is true even in those regions that have been shown to interact with the C1q fragment of complement and the various FcγR classes. FIG. 1 provides a alignment of the amino acid sequences of Cγ1 and Cγ3. The other two isotypes of human IgG (Cγ2 and Cγ4) have sequence differences which have been associated with FcR binding. FIG. 2 provides a alignment of the amino acid sequences of Cγ1, Cγ2, and Cγ4. The important sequences for FcγR binding are Leu-Leu-Gly-Gly (residues 234 through 237 in Cγ1), located in the CH2 domain adjacent to the hinge. Canfield and Morrison, J. Exp. MED. 173: 1483-1491 (1991). These sequence motifs are conserved in Cγ1 and Cγ3, in agreement with their similar biological properties, and possibly related to the similarity of pharmacokinetic behavior when used to construct IL-2 fusion proteins. Many mutational analyses have been done to demonstrate the effect of specific mutations on FcR binding, including those in residues 234-237 as well as the hinge-proximal bend residue Pro₃₃₁ that is substituted by Ser in IgG4. Another important structural component necessary for effective FcR binding is the presence of an N-linked carbohydrate chain covalently bound to Asn₂₉₇. Enzymatic removal of this structure or mutation of the Asn residue effectively abolish, or at least dramatically reduce, binding to all classes of FcγR.

Brumbell et al. postulated the existence of a protection receptor (FcRp) that would slow the rate of catabolism of circulating antibodies by binding to the Fc portion of antibodies and, following their pinocytosis into cells, would redirect them back into the circulation. Brumbell et al., NATURE 203: 1352-1355 (1964). The beta-2 microglobulin-containing neonatal intestinal transport receptor (FcRn) has recently been identified as a FcRp. See, Junghans et al., PROC. NATL. ACAD. SCI. USA 93: 5512-5516 (1996). The sequences necessary for binding to this receptor are conserved in all four classes of human IgG and are located at the interface between the CH2 and CH3 domains. See, Medesan et al., J. IMMUNOL. 158: 2211-2217 (1997). These sequences have been reported to be important for the in vivo circulating half-life of antibodies. See, International PCT publication WO 97/34631. Thus, preferred antibody-based fusion proteins of the present invention will have the sequences necessary for binding to FcRp.

Methods for synthesizing useful embodiments of the invention are described, as well as assays useful for testing their pharmacokinetic activities, both in vitro and in pre-clinical in vivo animal models. The preferred gene construct encoding a chimeric chain includes, in 5′ to 3′ orientation, a DNA segment which encodes at least a portion of an immunoglobulin and DNA which encodes a second, non-immunoglobulin protein. An alternative preferred gene construct includes, in 5′ to 3′ orientation, a DNA segment which encodes a second, non-immunoglobulin protein and DNA which encodes at least a portion of an immunoglobulin. The fused gene is assembled in or inserted into an expression vector for transfection of the appropriate recipient cells where it is expressed.

The invention is illustrated further by the following non-limiting examples:

### EXAMPLE 1

### Improving the In Vivo Circulating Half-Life of an Antibody-IL2 Fusion Protein by Class Switching from Cγ1 to Cγ4 IgG Constant Regions

According to the present invention, antibody-based fusion proteins with enhanced in vivo circulating half-lives can be obtained by constructing antibody-based fusion proteins using sequences from antibody isotypes that have reduced or no binding affinity for Fc receptors.

In order to assess whether the in vivo circulating half-life of the antibody-based fusion protein can be enhanced by using sequences from antibody isotypes with reduced or no binding affinity for Fc receptors, an antibody-IL2 fusion protein with a human Cγ1 constant region (Fc region) was compared to an antibody-IL2 fusion protein with a human Cγ4 Fc region.

1.1 Construction of Antibody-IL2 Fusion Proteins with a Cγ4 IgG Constant Region

The construction of antibody-IL2 fusion proteins with a Cγ1 constant region has been described in the prior art. See, for example, Gillies et al., PROC. NATL. ACAD. SCI. USA 89: 1428-1432 (1992); and U.S. Pat. No 5,650,150, the disclosure of which is incorporated herein by reference.

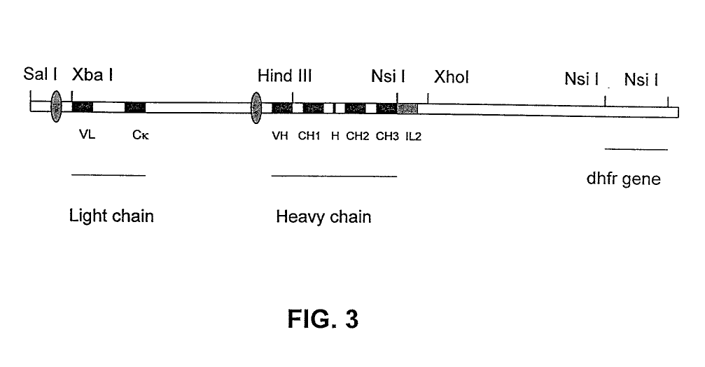

To construct antibody-IL2 fusion proteins with a Cγ4 constant region, a plasmid vector, capable of expressing a humanized antibody-IL2 fusion protein with variable (V) regions specific for a human pancarcinoma antigen (KSA) and the human Cγ1 heavy chain fused to human IL-2, was modified by removing the Cγ1 gene fragment and replacing it with the corresponding sequence from the human Cγ4 gene. A map of some of the relevant restriction sites and the site of insertion of the Cγ4 gene fragment is provided in FIG. 3. These plasmid constructs contain the cytomegalovirus (CMV) early promoter for transcription of the mRNA encoding the light (L) and heavy (H) chain variable (V) regions derived from the mouse antibody KS-¼. The mouse V regions were humanized by standard methods and their encoding DNA sequences were chemically synthesized. A functional splice donor site was added at the end of each V region so that it could be used in vectors containing H and L chain constant region genes. The human CK light chain gene was inserted downstream of the cloning site for the VL gene and was followed by its endogenous 3′ untranslated region and poly adenylation site. This transcription unit was followed by a second independent transcription unit for the heavy chain-IL2 fusion protein. It is also driven by a CMV promoter. The VH encoding sequence was inserted upstream of the DNA encoding the Cγ heavy chain gene of choice, fused to human IL-2 encoding sequences. Such Cγ genes contain splice acceptor sites for the first heavy chain exon (CH1), just downstream from a unique Hind III common to all human Cγ genes. A 3′ untranslated and polyadenylation site from SV40 virus was inserted at the end of the IL-2 encoding sequence. The remainder of the vector contained bacterial plasmid DNA necessary for propagation in E. coli and a selectable marker gene (dihydrofolate reductase—dhfr) for selection of transfectants of mammalian cells.

The swapping of the Cγ1 and Cγ4 fragments was accomplished by digesting the original Cγ1-containing plasmid DNA with Hind III and Xho I and purifying the large 7.8 kb fragment by agarose gel electrophoresis. A second plasmid DNA containing the Cγ4 gene was digested with Hind III and Nsi I and the 1.75 kb fragment was purified. A third plasmid containing the human IL-2 cDNA and SV40 poly A site, fused to the carboxyl terminus of the human Cγ1 gene, was digested with Xho I and Nsi I and the small 470 bp fragment was purified. All three fragments were ligated together in roughly equal molar amounts and the ligation product was used to transform competent E. coli. The ligation product was used to transform competent E. coli and colonies were selected by growth on plates containing ampicillin. Correctly assembled recombinant plasmids were identified by restriction analyses of plasmid DNA preparations from isolated transformants and digestion with Fsp I was used to discriminate between the Cγ1 (no Fsp I) and Cγ4 (one site) gene inserts. The final vector, containing the Cγ4-IL2 heavy chain replacement, was introduced into mouse myeloma cells and transfectants were selected by growth in medium containing methotrexate (0.1 μM). Cell clones expressing high levels of the antibody-IL2 fusion protein were expanded and the fusion protein was purified from culture supernatants using protein A Sepharose chromatography. The purity and integrity of the Cγ4 fusion protein was determined by SDS-polyacrylamide gel electrophoresis. IL-2 activity was measured in a T-cell proliferation assay and found to be identical to that of the Cγ1 construct.

1.2 Binding to Fc Receptors by Antibody and Antibody-IL2 Fusion Proteins with Cγ1 and Cγ4 IgG Constant Region.

Various mouse and human cell lines express one or more Fc receptor. For example, the mouse J774 macrophage-like cell line expresses FcRγI that is capable of binding mouse or human IgG of the appropriate subclasses. Likewise, the human K562 erythroleukemic cell line expresses FcRγII but not FcRγI. In order to assess the potential contribution of Fc receptor binding to clearance of antibody-based fusion proteins from the circulation, the binding affinities of an antibody, a Cγ1-IL2 fusion protein, and a Cγ4-IL2 fusion protein for FcRγI were compared in the mouse J774 cell line.

The two antibody-IL-2 fusion proteins described in Example 1, hu-KSγ1-IL2 and hu-KSγ4-IL2, were diluted to 2 μg/ml in PBS containing 0.1% bovine serum albumin (BSA), together with 2×10⁵ J774 cells in a final volume of 0.2 ml. After incubation on ice for 20 min, a FITC-conjugated anti-human IgG Fc antibody (Fab₂) was added and incubation was continued for an additional 30 min. Unbound antibodies were removed by two washes with PBS-BSA, and the cells were analyzed in a fluorescence-activated cell sorter (FACS). Control reactions contained the same cells mixed with just the FITC-labeled secondary antibody or with the humanized KSγ1 antibody (without IL-2).

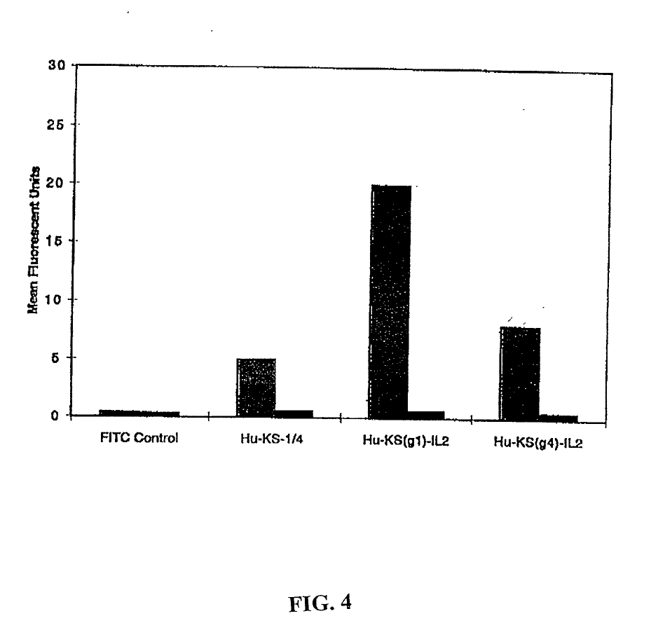

As expected, the binding of the Cγ4-IL2 fusion protein to J774 cells was significantly lower than the binding of the Cγ1-IL2 fusion protein. See FIG. 4. Unexpectedly, however, both the Cγ1-IL2 and Cγ4-IL2 fusion proteins had significantly higher binding to J774 cells than the KSγ1 antibody (without IL-2). This suggests that fusing a second protein, such as a cytokine, to an immunoglobulin may alter the antibody structure, resulting in an increase in binding affinity for one or more of the cell-bound Fc receptors, thereby leading to a rapid clearance from the circulation.

In order to determine whether the greater binding observed with IL-2 fusion proteins was due to the presence of IL-2 receptors or FcRγI receptors on the cells, excess mouse IgG (mIgG) was used to compete the binding at the Fc receptors. As illustrated in FIG. 4, background levels of binding were observed with the antibody and both antibody-IL2 fusion proteins in the presence of a 50-fold molar excess of mIgG. This suggests that the increased signal binding of antibody-IL2 fusion proteins was due to increased binding to the Fc receptor.

Cell lines expressing Fc receptors are useful for testing the binding affinities of candidate fusion proteins to Fc receptors in order to identify antibody-based fusion proteins with enhanced in vivo half lives. Candidate antibody-based fusion proteins can be tested by the above-described methods. Candidate antibody-based fusion proteins with substantially reduced binding affinity for an Fe receptor will be identified as antibody-based fusion proteins with enhanced in vivo half lives.

1.3 Measuring the Circulating Half-Life of Antibody-IL2 Fusion Proteins with Cγ1 and Cγ4 IgG constant region.

In order to assess whether using the Fc region of an IgG isotype having reduced affinity for Fc receptors will enhance the in vivo circulating half-life, fusion proteins containing the Cγ1 isotype heavy chain (i. e., hu-KSγ1-IL2) were compared to fusion proteins containing the Cγ4 isotype heavy chain (i.e., hu-KSγ4-IL2).

Purified humanized KS-¼-IL2 fusion proteins containing either the Cγ1 or Cγ4 isotype heavy chain were buffer-exchanged by diafiltration into phosphate buffered saline (PBS) and diluted further to a concentration of ˜100 μg/ml. Approximately 20 μg of the antibody-based fusion protein (0.2 ml) was injected into 6-8 week old Balb/c mice in the tail vein using a slow push. Four mice were injected per group. At various time points, small blood samples were taken by retro-orbital bleeding from anaesthetized animals and collected in tubes containing citrate buffer to prevent clotting. Cells were removed by centrifugation in an Eppendorf high-speed tabletop centrifuge for 5 min. The plasma was removed with a micropipettor and frozen at −70° C. The concentration of human antibody determinants in the mouse blood was measured by ELISA. A capture antibody specific for human H and L antibody chains was used for capture of the fusion proteins from the diluted plasma samples. After a two hour incubation in antibody-coated 96-well plates, the unbound material was removed by three washes with ELISA buffer (0.01% Tween 80 in PBS). A second incubation step used either an anti-human Fc antibody (for detection of both antibody and intact fusion protein), or an anti-human IL-2 antibody (for detection of only the intact fusion protein). Both antibodies were conjugated to horse radish peroxidase (HRP). After a one hour incubation, the unbound detecting antibody was removed by washing with ELISA buffer and the amount of bound HPR was determined by incubation with substrate and measuring in a spectrophotometer.

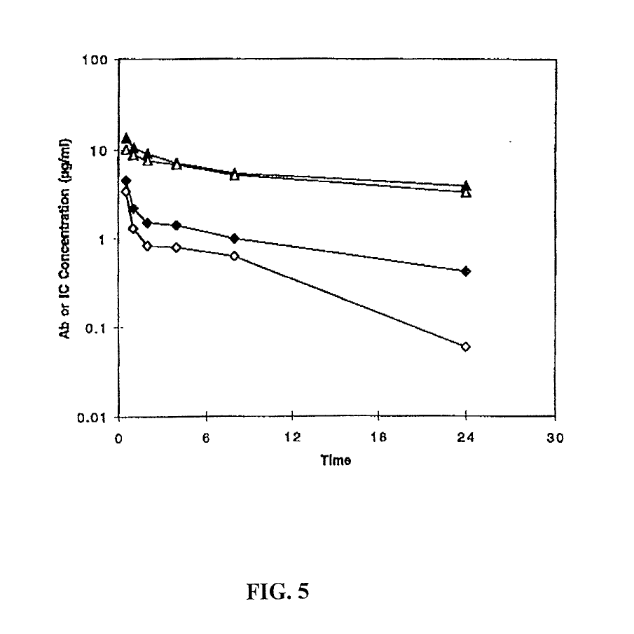

As depicted in FIG. 5, the (x phase half-life of the hu-KSγ4-IL2 fusion protein was significantly longer than the α phase half-life of the hu-KSγ1-IL2 fusion protein. The increased half-life is best exemplified by the significantly higher concentrations of the hu-KSγ4-IL2 fusion protein (3.3 μg/ml) compared to the hu-KSγ1-IL2 fusion protein (60 ng/ml) found in mice after 24 hours.

The hu-KSγ1-IL2 protein had a rapid distribution (α) phase followed by a slower catabolic (β) phase, as reported earlier for the chimeric 14.18-IL2 fusion protein. See, Gillies et al., BIOCONJ. CHEM. 4: 230-235 (1993). In the Gillies et al. study, only antibody determinants were measured, so it was not clear if the clearance represented the clearance of the intact fusion protein or the clearance of the antibody component of the fusion protein. In the present Example, samples were assayed using both (1) an antibody-specific ELISA, and (2) a fusion protein-specific ELISA (i.e., an ELISA that requires that both the antibody and IL-2 components be physically linked). As illustrated in FIG. 5, in animals injected with the hu-KSγ1-IL2 fusion protein, the amount of circulating fusion protein was lower than the total amount of circulating antibody, especially at the 24 hr time point. This suggests that the fusion protein is being proteolytically cleaved in vivo and that the released antibody continues to circulate. Surprisingly, in animals injected with the hu-KSγ4-IL2 fusion protein, there was no significant differences between the amount of circulating fusion protein and the total amount of circulating antibody. This suggests the hu-KSγ4-IL2 fusion protein was not being proteolytically cleaved in these animals during the 24 hour period measured.

As discussed above, Cγ1 and Cγ3 have binding affinity for Fc receptors, whereas while Cγ4 has reduced binding affinity and Cγ2 has no binding affinity for Fc receptors. The present Example described methods for producing antibody-based fusion proteins using the Cγ4 Fe region, an IgG isotype having reduced affinity for Fe receptors, and established that such antibody-based fusion proteins have enhanced in vivo circulating half-life. Accordingly, a skilled artisan can use these methods to produce antibody-based fusion proteins with the Cγ2 Fe region, instead of the Cγ4 Fe region, in order to enhance the circulating half-life of fusion proteins. A Hu-KS-IL2 fusion protein utilizing the human Cγ2 region can be constructed using the same restriction fragment replacement and the above-described methods for Cγ4-IL2 fusion protein. and tested using the methods described herein to demonstrate increased circulating half-life. Antibody-based fusion proteins with the Cγ2 Fe region, or any other Fe region having reduced binding affinity or lacking binding affinity for a Fe receptor will have enhanced in vivo circulating half-life compared to antibody-based fusion proteins having binding affinity for a Fe receptor.

### EXAMPLE 2

### Mutating the Human Cγ1 or Cγ3 Gene in Antibody-Based Fusion Protein Constructs to Improve Their In Vivo Circulating Half-Life

IgG molecules interact with several molecules in the circulation, including members of the complement system of proteins (e.g., C1q fragment), as well as the three classes of FcR. The important residues for C1q binding are residues Glu₃₁₈, Lys₃₂₀, and Lys₃₂₂ which are located in the CH2 domains of human heavy chains. Tao et al., J. EXP. MED. 178: 661-667 (1993). In order to discriminate between FcR and C1q binding as mechanisms for rapid clearance, we substituted the more drastically altered Cγ2 hinge-proximal segment into the Cγ1 heavy chain. This mutation is expected to affect FcR binding but not complement fixation.

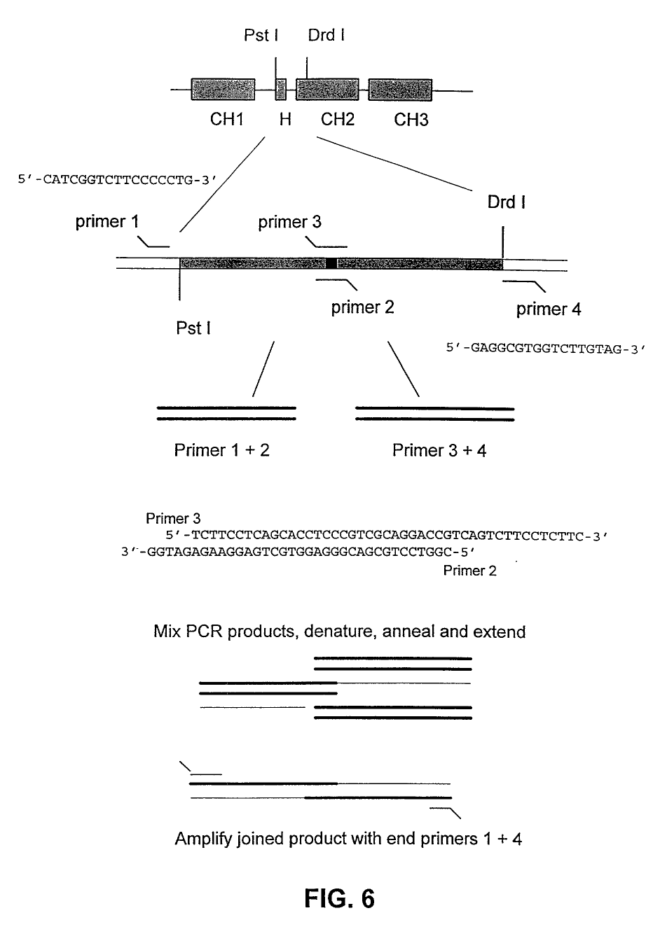

The mutation was achieved by cloning and adapting the small region between the hinge and the beginning of the CH2 exon of the germ line Cγ1 gene using overlapping polymerase chain reactions (PCR). The PCR primers were designed to substitute the new sequence at the junction of two adjacent PCR fragments spanning a Pst I to Drd I fragment (see FIG. 6). In the first step, two separate PCR reactions with primers 1 and 2 (SEQ ID NOS: 5 and 6, respectively), or primers 3 and 4 (SEQ ID NOS: 7 and 8, respectively), were prepared using the Cγ1 gene as the template. The cycle conditions for the primary PCR were 35 cycles of: 94° C. for 45 sec, annealing at 48° C. for 45 seconds, and primer extension at 72° C. for 45 sec. The products of each PCR reaction were used as template for the second, joining reaction step. One tenth of each primary reaction was mixed together and combined with primers 1 and 4 to amplify only the combined product of the two initial PCR products. The conditions for the secondary PCR were: 94° C. for 1 min, annealing at 51° C. for 1 min, and primer extension at 72° C. for 1 min. Joining occurs as a result of the overlapping between the two individual fragments which pairs with the end of the other, following denaturation and annealing. The fragments that form hybrids get extended by the Taq polymerase, and the complete, mutated product was selectively amplified by the priming of the outer primers, as shown in FIG. 6. The final PCR product was cloned in a plasmid vector and its sequence verified by DNA sequence analysis.

The assembly of the mutated gene was done in multiple steps. In the first step, a cloning vector containing the human Cγ1 gene was digested with Pst I and Xho I to remove the non-mutated hinge-CH2-CH3 coding sequences. A Drd I to Xho I fragment encoding part of CH2, all of CH3 and the fused human IL-2 coding sequences was prepared from the Cγ1-IL2 vector, described above. A third fragment was prepared from the subcloned PCR product by digestion with Pst I and Drd I. All three fragments were purified by agarose gel electrophoresis and ligated together in a single reaction mixture. The ligation product was used to transform competent E. coli and colonies were selected by growth on plates containing ampicillin. Correctly assembled recombinant plasmids were identified by restriction analyses of plasmid DNA preparations from isolated transformants and mutated genes were confirmed by DNA sequence analysis. The Hind III to Xho I fragment from the mutated Cγ1-IL2 gene was used to reassemble the complete hu-KS antibody-IL2 fusion protein expression vector.

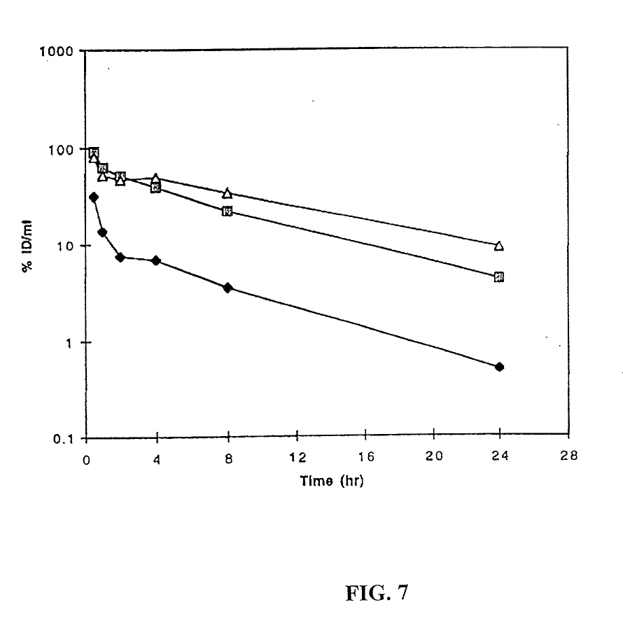

In order to assess the enhancement of the in vivo circulating half-life induced by a mutation of an important amino acid for FcR binding, and to discriminate between FcR and C1q binding as mechanisms for rapid clearance, the in vivo plasma concentration of the mutated hu-KSγ1-IL2 was compared to the plasma concentration of hu-KSγ1-IL2 at various specified times. As illustrated in FIG. 7, the in vivo clearance rates of the mutated hu-KSγ1-IL2 and hu-KSγ4-IL2 were significantly lower than the clearance rate of hu-KSγ1-IL2. These results suggests that an antibody-based fusion protein with enhanced in vivo circulating half-life can be obtained by modifying sequences necessary for binding to Fc receptors in isotypes that have binding affinity for an Fc receptor. Further, the results suggests that the mechanisms for rapid clearance involve FcR binding rather than C1q binding.

The skilled artisan will understand, from the teachings of the present invention, that several other mutations to the Cγ1 or Cγ3 genes can be introduced in order to reduce binding to FcR and enhance the in vivo circulating half-life of an antibody-based fusion protein. Moreover, mutations can also be introduced into the Cγ4 gene in order to further reduce the binding of Cγ4 fusion proteins to FcR. For example, additional possible mutations include mutations in the hinge proximal amino acid residues, mutating Pro₃₃₁, or by mutating the single N-linked glycosylation site in all IgG Fc regions. The latter is located at Asn₂₉₇ as part of the canonical sequence: Asn-X-Thr/Ser, where the second position can be any amino acid (with the possible exception of Pro), and the third position is either Thr or Ser. A conservative mutation to the amino acid G1n, for example, would have little effect on the protein but would prevent the attachment of any carbohydrate side chain. A strategy for mutating this residue might follow the general procedure, just described, for the hinge proximal region. Methods for generating point mutations in cloned DNA sequences are well established in the art and commercial kits are available from several vendors for this purpose.

### EXAMPLE 3

### Increasing the Circulating Half-Life of Receptor-Antibody-Based Fusion Proteins

Several references have reported that the Fc portion of human IgG can serve as a useful carrier for many ligand-binding proteins, or receptors, with biological activity. Some of these ligand-binding proteins have been fused to the N-terminal of the Fc portion of an Ig, such as CD4, CTLA-4, and TNF receptors. See, for example, Capon et al., NATURE 337: 525-531 (1989); Linsley et al., J. EXP. MED. 174: 561-569 (1991); Wooley et al., J. IMMUNOL. 151. 6602-6607 (1993). Increasing the circulating half-life of receptor-antibody-based fusion proteins may permit the ligand-binding protein partner (i.e., the second non-Ig protein) to more effectively (1) block receptor-ligand interactions at the cell surface; or (2) neutralize the biological activity of a molecule (e.g., a cytokine) in the fluid phase of the blood, thereby preventing it from reaching its cellular target. In order to assess whether reducing the ability of receptor-antibody-based fusion proteins to bind to IgG receptors will enhance their in vivo circulating half-life, receptor-antibody-based fusion proteins with human Cγ1 Fc regions are compared to antibody-based fusion proteins with human Cγ4 Fc regions.

To construct CD4-antibody-based fusion proteins, the ectodomain of the human CD4 cell surface receptor is cloned using PCR from human peripheral blood monocytic cells (PBMC). The cloned CD4 receptor includes compatible restriction sites and splice donor sites described in Example 1. The expression vector contains a unique Xba I cloning site downstream of the CMV early promoter, and the human Cγ1 or Cγ4 gene downstream of their endogenous Hind III site. The remainder of the plasmid contains bacterial genetic information for propagation in E. coli, as well as a dhfr selectable marker gene. Ligated DNAs are used to transform competent bacteria and recombinant plasmids are identified from restriction analyses from individual bacterial colonies. Two plasmid DNA constructs are obtained: CD4-Cγ1 and CD4-Cγ4.

The expression plasmids are used to transfect mouse myeloma cells by electroporation and transfectants are selected by growth in culture medium containing methotrexate (0.1 μM). Transfectants expressing the fusion proteins are identified by ELISA analyses and are expanded in culture in order to generate fusion protein for purification by binding to and elution from protein A Sepharose. Purified proteins in chromatography elution buffer are diafiltered into PBS and diluted to a final concentration of 100 μg/ml. Balb/c mice are injected with 0.2 ml (20 μg) of either the CD4-Cγ1 or CD4-Cγ4 fusion protein and the pharmacokinetics are tested as described in Example 1.3. The CD4-Cγ4 fusion protein has a significantly greater half-life than the CD4-Cγ1 fusion protein.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. An antibody-based fusion protein with an enhanced circulating half-life, comprising at least a portion of an immunoglobulin (Ig) heavy chain having substantially reduced binding affinity for an Fc receptor, said portion of heavy chain being linked to a second non-Ig protein, said antibody-based fusion protein having a longer circulating half-life in vivo than an unlinked second non-Ig protein.

2. The antibody-based fusion protein of claim 1, wherein said portion of heavy chain comprises at least the CH2 domain of an IgG2 or IgG4 constant region.

3. The antibody-based fusion protein of claim 1, wherein said portion of heavy chain comprises at least a portion of an IgG1 constant region having a mutation or a deletion at one or more amino acid selected from the group consisting of Leu₂₃₄, Leu₂₃₅, Gly₂₃₆, Gly₂₃₇, Asn₂₉₇, and Pro₃₃₁.

4. The antibody-based fusion protein of claim 1, wherein said portion of heavy chain comprises at least a portion of an IgG3 constant region having a mutation or a deletion at one or more amino acid selected from the group consisting of Leu₂₈₁, Leu₂₈₂, Gly₂₈₃, Gly₂₈₄, Asn₃₄₄, and Pro₃₇₈.

5. The antibody-based fusion protein of claim , wherein said portion of heavy chain further has binding affinity for an immunoglobulin protection receptor.

6. The antibody-based fusion protein of claim 1, wherein said portion of heavy chain has substantially reduced binding affinity for a Fc receptor selected from the group consisting of FcγRI, FcγRII and FcγRIII.

7. The antibody-based fusion protein of claim 1, wherein said second non-Ig protein is selected from the group consisting of a cytokine, a ligand-binding protein, and a protein toxin.

8. The antibody-based fusion protein of claim 1, wherein said cytokine is selected from the group consisting of a tumor necrosis factor, an interleukin, and a lymphokine.

9. The antibody-based fusion protein of claim 8, wherein said tumor necrosis factor is tumor necrosis factor alpha.

10. The antibody-based fusion protein of claim 8, wherein said interleukin is interleukin-2.

11. The antibody-based fusion protein of claim 8, wherein said lymphokine is a lymphotoxin or a colony stimulating factor.

12. The antibody-based fusion protein of claim 11, wherein said colony stimulating factor is a granulocyte-macrophage colony stimulating factor.

13. The antibody-based fusion protein of claim 1, wherein said ligand-binding protein is selected from the group consisting of CD4, CTLA-4, TNF receptor, and an interleukin receptor.

14. A method of increasing the circulating half-life of an antibody-based fusion protein, comprising the step of linking at least a portion of an Ig heavy chain to a second non-Ig protein, said portion of heavy chain having substantially reduced binding affinity for an Fc receptor, thereby forming an antibody-based fusion protein having a longer circulating half-life in vivo than an unlinked second non-Ig protein.

15. The method of claim 14, wherein said portion of heavy chain comprises at least the CH2 domain of an IgG2 or IgG4 constant region.

16. A method of increasing the circulating half-life of an antibody-based fusion protein, comprising the steps of:
(a) introducing a mutation or a deletion at one or more amino acid of an IgG1 constant region, said amino acid selected from the group consisting of Leu₂₃₄, Leu₂₃₅, Gly₂₃₆, Gly₂₃₇, Asn₂₉₇, and Pro₃₃₁, thereby producing an Ig heavy chain having substantially reduced binding affinity for an Fc receptor; and
(b) linking at least a portion of the heavy chain of step (a) to a second non-Ig protein,
thereby forming an antibody-based fusion protein having a longer circulating half-life in vivo than an unlinked second non-Ig protein.

17. A method of increasing the circulating half-life of an antibody-based fusion protein, comprising the steps of:
(a) introducing a mutation or a deletion at one or more amino acid of an IgG3 constant region, said amino acid selected from the group consisting of Leu₂₈₁, Leu₂₈₂, Gly₂₈₃, Gly₂₈₄, Asn₃₄₄, and Pro₃₇₈, thereby producing an Ig heavy chain having substantially reduced binding affinity for an Fc receptor; and
(b) linking at least a portion of the Ig heavy chain of step (a) to a second non-Ig protein,
thereby forming an antibody-based fusion protein having a longer circulating half-life in vivo than an unlinked second non-Ig protein.

18. The method of claim 14, 16 or 17, wherein said portion of heavy chain further has binding affinity for an immunoglobulin protection receptor.

19. The method of claim 14, 16 or 17, wherein said portion of heavy chain has substantially reduced binding affinity for a Fc receptor selected from the group consisting of FcγRI, FcγRII and FcγRIII.

20. The method of claim 14, 16 or 17, wherein said second non-Ig protein is selected from the group consisting of a cytokine, a ligand-binding protein, and a protein toxin.

21. The method of claim 14, 16 or 17, wherein said cytokine is selected from the group consisting of a tumor necrosis factor, an interleukin, and a lymphokine.

22. The method of claim 21, wherein said tumor necrosis factor is tumor necrosis factor alpha.

23. The method of claim 21, wherein said interleukin is interleukin-2.

24. The method of claim 21, wherein said lymphokine is a lymphotoxin or a colony stimulating factor.

25. The antibody-based fusion protein of claim 24, wherein said colony stimulating factor is a granulocyte-macrophage colony stimulating factor.

26. The method of claim 14, 16 or 17, wherein said ligand-binding protein is selected from the group consisting of CD4, CTLA-4, TNF receptor, and an interleukin receptor.


SEQUENCE LISTING (8 sequences)

SEQ ID NO: 1 - protein, 447 aa, Homo sapiens
  IGG-1 CHAIN C REGION
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 443 aa, Homo sapiens
  IGG-2 CHAIN C REGION
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 494 aa, Homo sapiens
  IGG-3 CHAIN C REGION
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 444 aa, Homo sapiens
  IGG-4 CHAIN C REGION
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 17 bp, Artificial Sequence
  Description of Artificial Sequence primer 1
catcggtcttccccctg

SEQ ID NO: 6 - DNA, 35 bp, Artificial Sequence
  Description of Artificial Sequence primer 2
cggtcctgcgacgggaggtgctgaggaagagatgg

SEQ ID NO: 7 - DNA, 45 bp, Artificial Sequence
  Description of Artificial Sequence primer 3
tcttcctcagcacctcccgtcgcaggaccgtcagtcttcctcttc

SEQ ID NO: 8 - DNA, 17 bp, Artificial Sequence
  Description of Artificial Sequence primer 4
gaggcgtggtcttgtag